(19) United States
(12) Patent Application Publication
(10) Pub. No.: US 2012/0022163 A1
(43) Pub. Date: Jan. 26, 2012

(54) 1-(2-FLUOROBIPHENYL-4-YL)-CYCLOPROPANECARBOXYLIC ACID DERIVATIVES FOR THE THERAPY OF PRION DISEASES

(75) Inventors: Bruno Pietro Imbimbo, Parma (IT); Gino Villetti, Parma (IT); Giorgio Poli, Parma (IT)

(73) Assignee: Chiesi Farmaceutici S.p.A., Parma (IT)

(21) Appl. No.: 13/153,598

(22) Filed: Jun. 6, 2011

(30) Foreign Application Priority Data
Jun. 4, 2010 (EP) 10164967.1

(51) Int. Cl. A61K 31/192; A61P 25/00; A61P 31/00

(52) U.S. Cl. 514/570

(57) ABSTRACT

Derivatives of 1-(2-fluorobiphenyl-4-yl)-cyclopropanecarboxylic acid are useful for the prevention and/or treatment of prion diseases in animals and humans.

### CROSS REFERENCES TO RELATED APPLICATIONS

This application claims priority to European Patent Application No. 10164967.1 filed on Jun. 4, 2010, which is incorporated herein by reference in its entirety.

### BACKGROUND OF THE INVENTION

1. Field of the Invention

The present invention relates to methods for the prevention and/or treatment of prion diseases.

2. Discussion of the Background

Transmissible Spongiform Encephalopathies (TSEs) are a group of rare and fatal neurodegenerative diseases of humans and animals, characterized by the presence of amyloid plaques, gliosis, vacuolization and neuronal death by apoptosis in the central nervous system (CNS). At present the prion is considered the causal agent of these diseases; it is an infectious agent consisting of an unconventional abnormal isoform (PrP^{sc}) of a protein (PrP^{c}) normally present in brain cells, accumulating in the CNS because of its resistance to endogenous proteases. For this reason, TSEs are also known as prion diseases.

Prion diseases may occur as sporadic forms, inherited forms, associated with mutations within the prion protein gene (PRNP), and acquired forms, by oral or iatrogenic transmission of the prion.

The most common human prion disease is the Creutzfeldt-Jakob disease (CJD).

The sporadic form generally occurs in the seventh decade or later and has a typically short course (average 4 to 6 months), while inherited (genetic) form usually starts at a younger age and has a more protracted course.

In humans, prion diseases occur worldwide with an incidence of roughly 1 per 10⁶ populations per year for sporadic disease and 1 per 10⁷⁻⁸ per year for inherited disease.

The new CJD, that affected young people (mean age: 26 years), referred as Variant Creutzfeldt-Jakob disease (vCJD) is apparently associated with the consumption of infected tissue from BSE (Bovine Spongiform Encephalopathies) infected cattle.

All prion diseases are fatal and yet, there are no approved drugs capable of preventing or reversing said diseases.

A particular focus of previous research and development efforts was on preventing formation of synaptotoxic β-amyloid (Aβ) peptide in the brain and its aggregation into plaques.

Since protein misfolding and deposition of amyloid in the CNS are pathogenic characteristics shared by prion diseases and Alzheimer's disease, drugs proposed for the treatment of the latter disease such as gamma-secretase inhibitors have also been proposed for the therapy of prion diseases.

However, due to their inhibitory activity on the cleavage of the Notch-1 protein, safety concerns have been raised on the therapeutic use of such a class of compounds.

Derivatives of 1-(2-fluorobiphenyl-4-yl)-cyclopropanecarboxylic acid for the treatment of Alzheimer's disease have been first described in patent application WO 2004/074232 as one of different class of candidate therapeutic agents for neurodegenerative diseases such as Alzheimer's disease. In particular, the compound 1-(3′,4′-dichloro-2-fluorobiphenyl-4-yl)cyclopropanecarboxylic acid has been found to act as a gamma secretase modulator. It has also been quoted with the experimental code CHF 5074. In WO 2008/36733, said compound is generically cited among a large number of potential therapeutic agents for the treatment of vescicle transport disorders.

### SUMMARY OF THE INVENTION

Accordingly, it is one object of the present invention to provide novel methods for the prevention and/or treatment of prion diseases.

These and other objects, which will become apparent during the following detailed description, have been achieved by the inventors' discovery that 1-(3′,4′-dichloro-2-fluorobiphenyl-4-yl)cyclopropanecarboxylic acid (CHF 5074) significantly increases the survival in an animal model of mice experimentally infected with the causative agent of a prion disease. Therefore, CHF 5074 and related compounds can be advantageously utilized for the prevention and/or treatment of a prion disease, in particular for delaying the onset and/or slowing the progression in sporadic and/or acquired (dietary and iatrogenic) forms of prion diseases.

According to the above aspects, the present invention provides a method for the prevention and/or treatment of a prion disease by administering to a subject in need thereof an effective amount of a compound of formula (I):

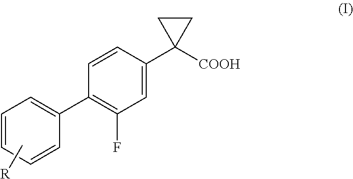

wherein R represents one or more groups, which can be the same or different from each other, independently selected from halogen atoms, preferably chlorine.

Preferably, the compound of formula (I) is 1-(3′,4′-dichloro-2-fluorobiphenyl-4-yl)cyclopropanecarboxylic acid also known with the code CHF 5074.

The present invention is also directed to the use of the compounds of general formula (I) in the manufacture of a medicament for the prevention and/or treatment of a prion disease.

In another aspect, the present invention is also directed to the use of polymorphs, pharmaceutically acceptable salts and prodrugs thereof.

In a further aspect, the present invention provides a method for preventing and/or treating a prion disease in a patient, comprising administering an effective amount of a compound of general formula (I), including polymorphs, pharmaceutically acceptable salts and prodrugs thereof.

### BRIEF DESCRIPTION OF THE DRAWINGS

A more complete appreciation of the invention and many of the attendant advantages thereof will be readily obtained as the same become better understood by reference to the following detailed description when considered in connection with the accompanying drawings, wherein:

FIG. 1 shows the survival probability of ip infected and CHF 5074 treated animals versus ip infected but untreated animals.

FIG. 2 shows the mean lesion profile in the animals infected by intraperitoneal route (ip) treated and untreated versus the control animals.

FIG. 3 shows the mean quantification scores of PrP^{sc} deposition in cerebellum, hippocampus and parietal cortex of intraperitoneally infected mice treated with vehicle or CHF5074. Columns indicate mean severity score of PrP^{sc} staining by immunohistochemistry. Error bars represent the standard error of the means.

### DETAILED DESCRIPTION OF THE PREFERRED EMBODIMENTS

The term “prion” refers to a small proteinaceous infectious particle that resists inactivation by treatments that modify nucleic acids.

The expression “a prion disease caused by infection” means that the prion enters the body either from the diet or following medical procedures (such as surgery, growth hormone injections, and corneal transplants).

The expression “a prion disease of genetic cause” means a disease of apparent hereditary mendelian transmission. Where the prion disease is genetic, it is not prima facie consistent with an infectious agent.

Unless otherwise provided in the present description, within the compounds of general formula (I), the phenyl ring bears one or more halogen atoms therein referred to as R₃ groups.

From the above, it is clear to the skilled person that any of the said halogen atoms, the same or different from each other, may be thus present in any possible free position of the phenyl ring itself.

The term “halogen atoms” as used herein includes fluorine, chlorine, bromine, and iodine.

The term “polymorphs” refers to a different crystal structure of the same solid substance. They exhibit different melting points, solubilities (which affect the dissolution rate of the drug and consequently its bioavailability in the body), X-ray crystal and diffraction patterns.

The expression “substantially pure polymorph” refers to a sample in which the polymorph is present in a substantial excess over other polymorphs of the same compound, i.e. in an amount exceeding 75%, more preferably exceeding 90%, even more preferably exceeding 95%, and most preferably exceeding 99% by weight of the total weight of the compound in the sample.

The term “prodrug” refers to a substance administered in an inactive form that is then metabolized in the body in vivo into the active compound with the aim of optimizing absorption, distribution, metabolism, and excretion. In particular, in the context of the present application, prodrugs are utilized to improve the CNS drug level, with poor crossing of the blood brain barrier usually being the limiting factor.

The term “prevention” refers to the use for reducing the occurrence of the disease or reducing the likelihood of contracting the disease.

The term “treatment” refers to a therapeutic treatment including, but not limited to palliative, curing, symptom-allievating, symptom-reducing, progression-slowing, onset delaying treatments.

The invention is directed to the compounds of formula (I)

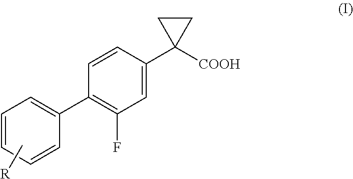

wherein R has the above reported meaning, and polymorphs, pharmaceutically acceptable salts and prodrugs thereof for use for the prevention and/or treatment of a prion disease.

Advantageously, R represents one or more chlorine atoms, and preferably the compound of formula (I) is 1-(3′,4′-dichloro-2-fluorobiphenyl-4-yl)cyclopropanecarboxylic acid, hereinafter quoted with the code CHF 5074.

The compounds of general formula (I) may be prepared according to the procedures described in the co-pending application WO 2009/149797, which is incorporated herein by reference in its entirety.

Said compounds may advantageously be used in any form, amorphous or crystalline and solvates or hydrates thereof. Preferably, they are used in crystalline form.

As disclosed in the co-pending European Patent Application No. EP 10158954.7, the entire content of which is incorporated herein by reference in its entirety, CHF 5074 can exist in three stable crystalline polymorphic forms. Accordingly the present invention includes the use of any of said polymorphs, either in substantially pure form or admixed in any proportion.

In view of the close relationship between the compounds of general formula (I) in free acid form and those on the form of salts, the present invention is also directed to the use of pharmaceutically acceptable salts thereof. Pharmaceutically acceptable salts according to the present invention include those formed with both common organic and inorganic bases.

In particular, when the preferred compound according to the invention is used, the salts disclosed in the co-pending European Patent Application No. EP 10158954.7, which is incorporated herein by reference in its entirety, may advantageously be utilized.

The compounds of general formula (I) may also be administered in form of prodrugs. Suitable prodrugs may be esters with common alcohols such as ethanol or polyalcohols such as sorbitol, with sugars such as glucose, or with sugar acids such as ascorbic acid. In particular, since in prion diseases, the CNS is the most severe affected tissue, prodrugs which are able of crossing the blood brain barrier such as those disclosed in WO 2006/016219, which is incorporated herein by reference in its entirety, may be advantageously utilized.

The compounds of formula (I), may be combined with one or more pharmaceutically acceptable carriers or excipients to provide suitable pharmaceutical compositions. The pharmaceutically acceptable carriers or excipients may be advantageously selected from the group consisting of diluents, wetting agents, emulsifying agents, binders, coatings, fillers, glidants, lubricants, disintegrants, preservatives, stabilizers, surfactants, pH buffering substances, flavoring agents and similar ones. Comprehensive guidance on pharmaceutical excipients is given in Remington's Pharmaceutical Sciences Handbook, XVII Ed. Mack Pub., N.Y., U.S.A., which is incorporated herein by reference.

The pharmaceutical compositions of the invention may be formulated for administration by any convenient route, e.g. by oral, parenteral, topical, inhalation, buccal, nasal, rectal, vaginal, transdermal administration. Suitable dosage forms can include tablets, capsules, caplets, lozenges, suppositories, solutions, emulsions, suspensions, syrups, ointments, creams, oils, and powders. Preferably, the pharmaceutical compositions of the invention will be administered orally using appropriate dosage forms, such as capsules, tablets, caplets, etc.

The dosage of the compounds of general formula (I) and of their salts and prodrugs can vary within wide limits depending on the nature of the disease to be treated, the type of patient, and the mode of administration. A person skilled in the art can determine a therapeutically effective amount for each patient and thereby define the appropriate dosage. When the preferred compound of the invention is administered by oral route to humans, a typical daily dosage might fall within the range of 10 mg to 2000 mg, preferably between 100 to 1000 mg, administered in a single or multiple daily dosage units. Thus, a single dose of the pharmaceutical preparations of the invention conveniently comprises between about 100 and 1000 mg of CHF 5074 or salt or prodrug thereof.

The compounds of the present invention may be of use in prevention and/or treatment of any prion disease. They may be also of use for delaying the onset or slowing the progression of said diseases.

Prion diseases could affect humans and other mammals. Humans diseases include: CJD (Creutzfeldt-Jacob Disease); vCJD (Variant Creutzfeldt-Jacob Disease); GSS (Gerstmann-Straussler-Scheinker) syndrome; FFI (Fatal Familial Insomnia); Kuru; and Alpers Syndrome.

Examples of diseases affecting other mammals include: Scrapie, which affects sheep and goats; TME (transmissible mink encephalopathy), which affects minks; CWD (chronic wasting disease), which affects mules, deer, and elk; and BSE (bovine spongiform encephalopathy), which affects cows.

Preferably, the compounds of the invention, and in particular CHF 5074, are utilized for the prevention or for delaying the onset or slowing the progression or for the treatment of a prion disease caused by infection and/or a sporadic form.

The present invention also provides methods of reducing the likelihood of contracting a prion disease. In such methods, it may be preferred to administer a compound of formula (I) to a subject who is at an elevated risk of contracting such a disease. Variant Creutzfeldt-Jakob disease may be caused by human transmission of BSE (bovine spongiform encephalopathy or mad cow disease), a prion disease of cattle. Accordingly, one group of humans at an elevated risk of contracting a prion disease are those which have been exposed to BSE. Other people develop a prion disease spontaneously as they get older (called sporadic CJD). In this case, the elderly may be considered a population at an elevated risk of contracting a prion disease. In this case, the term elderly includes people over 60 years old, over 65 years old, over 70 years old, over 75 years old, over 80 years old, over 85 years old, over 90 years old, and over 95 years old. Others are accidentally infected with prions as a result of medical or surgical procedures (since prions stick to metal instruments, are very resistant to sterilization and can also be passed in blood transfusions). Thus, patients undergoing surgery or blood transfusions may be considered another population at an elevated risk of contracting a prion disease. In addition, cattle which receive feed contaminated with prions, in particular contaminated with scrapie, the long established sheep prion disease, are a group of animals considered to have an elevated risk of contracting a prion disease.

Other features of the invention will become apparent in the course of the following descriptions of exemplary embodiments which are given for illustration of the invention and are not intended to be limiting thereof.

### EXAMPLES

### Example 1

The aim of this example is to assess the therapeutic and/or preventive activity of 1-(3′,4′-dichloro-2-fluorobiphenyl-4-yl)cyclopropanecarboxylic acid (CHF 5074) on a murine model experimentally infected with the causative agent of a prion disease.

### Animals and Tissue Collection.

91 CD1 female mice, aged 3 to 4 weeks and weighing 10 to 12 g, housed in a conditioned environment (22±1° C.., 55+5% relative humidity, 12 hour light/dark cycles) and fed ad libitum were used. The animals were randomly divided into groups depending on the route of infection (intracerebrally, ic, or intraperitoneally, ip) with the RML (Rocky Mountains Laboratories) strain of the mouse scrapie agent. A 10% (weight/volume) homogenate of RML-infected CD1 brain in sterile saline was diluted in sterile saline to a final concentration of 1%, and 50 μl or 25 μl of the suspension were injected intracerebrally (ic) and intraperitoneal (ip) respectively, as reported by Spilman et al., 2008, PNAS, 2008; 29(105):10595-10600, which is incorporated herein by reference in its entirety.

Among the animals of each infected group (ic or ip), two subgroups of 15 treated orally with CHF5074 and untreated mice were created (ic infected—treated, ic infected—untreated, ip infected—treated and ip infected—untreated). Similarly, the uninfected animals were divided into two subgroups of 8 animals which were treated and inoculated respectively ic or ip with the same volume of a 1% brain homogenate from uninfected CD1 mice (ic uninfected—treated and ip uninfected—treated). Furthermore, a negative control group was created. The adopted experimental design is reported in Table 1.

TABLE 1
Experimental design: groups of animals.

| Number of | Route of | Treatment with | |
|---|---|---|---|
| animals | infection | CHF5074 | Name |
| 15 | / | no | negative control |
| 15 | Ic | no | ic infected-untreated |
| 15 | Ip | no | ip infected-untreated |
| 15 | Ic | yes | ic infected-treated |
| 15 | Ip | yes | ip infected-treated |
| 8 | / | yes | ic uninfected-treated |
| 8 | / | yes | ip uninfected-treated |

The treatment, consisting of the oral administration of CHF 5074 (375 ppm/day) in medicated feed, started 13 days before scrapie infection. The untreated animals were fed with standard laboratory feed. In order to monitor the appearance and development of neurological signs, mice were observed daily. All mice were sacrificed at a standard clinical end point, basing on terminal scrapie symptoms established by Thackry et al., (Journal of Virology, 2002; 76(5): -2517) and Meeker et al., (The mouse model for scrapie. Inoculation, clinical scoring and histopathological techniques. Methods in Molecular Biology, vol. 299: Amyloid proteins: methods and protocols. Edited by E. M. Sigurdsson, Humana Press Inc, Totowa, N.J., 2005; pp. 309-323), both of which are incorporated herein by reference in their entireties. At necropsy, the cerebral hemispheres, the brain stem and the cerebellum were removed, then each brain was divided longitudinally and one part fixed in 10% formalin for histopathological and immunohistochemical analysis and the other one stored at −20° C.. for Western blot.

### Western Blot Analysis.

Ten percent (w/v) homogenates of each frozen brain were prepared in lysis buffer (10% N-lauroylsarcosine diluted in Tris Buffer Saline pH 7,4). After incubation for 20 to 30 minutes, they were clarified by centrifugation at 22000×g for 20 minutes at 10° C.. (Optima TL-CE, Beckman Coulter). A rate of 1 ml was removed from each supernatant and digested with proteinase K (pK, 40 μg/mol) for 1 hour at 37° C.. After digestion, 10 μl of pK inhibitor phenylmethanesulfonyl fluoride (PMSF, 100 mM) was added. The samples were then centrifuged at 215000×g for 1 hour at 10° C.. The pellets obtained were dissolved in 50 μl of Laemmli buffer. After boiling for 5 to 10 minutes at 99° C.., 10 μl of each extract (10 mg of tissue) were separated by sodium dodecyl-sulfate polyacrylamide gel electrophoresis on a 12% minigel and then transferred onto Polyvinylidene Fluoride (PVDF) membranes (Immobilion P; Millipore, Billerica, Mass.). Blots were blocked with TBS-BSA 5% and incubated at 4° C. overnight with the mouse monoclonal antibody SAF 70 diluted 1:1000, recognizing sequence within amino acids 142-160 (human numbering) (Spi Bio, Cayman Chemical, Ann Arbor, Mich.). The immunodetection was carried out with alkaline phosphatase-conjugated goat anti-mouse IgG, revealed by a chemiluminescent substrate (Immunostar, Bio-Rad). The films obtained were subjected to densitometric analysis.

### Histopathological and Immunohistochemical Analysis.

Following fixation, brains were coronally cut in five sections (medulla, pons and cerebellum, mid-brain, diencephalon, telencephalon) according to Fraser et al., J Comp Path, 1968; 78(3):301-311, which is incorporated herein by reference in its entirety. These samples were processed and embedded in paraffin wax according to standard histopathological procedures. The 3 μm-thick sections obtained from each hemisphere were placed on slides with positive electrostatic charge and left for 24 hours at 37° C. An hematoxylin-eosin staining was performed for each brain section.

Spongiosis in different encephalic areas (medulla, cerebellum, mid-brain, hypothalamus, thalamus, hippocampus, para terminal body, frontal cortex and parietal cortex) was evaluated by light microscopy and an intensity grade was assigned to the different pattern detected: absent (0), slight (1), moderate (2), marked (3), very marked (4).

Slides for immunohistochemical analysis were dewaxed and rehydrated by routine methods and then immersed in 98% formic acid for 15 minutes. After washing in water, the sections were autoclaved for 30 minutes at 121° C. in citrate buffer (pH 6,1) to unmask antigenic sites. Endogenous peroxidase activity was blocked in 3% hydrogen peroxide diluted in methanol for 20 minutes at room temperature, and samples were left overnight in distilled water at 2-8° C. To block non-specific tissue antigens, the sections were incubated with 2% horse blocking serum (pH 7,4) for 20 minutes at room temperature and then incubated for 1 hour at room temperature with the mouse monoclonal antibody ICSM35 diluted 1:1000, recognizing sequence 93-102 of human PrP (D-Gen, London, UK). After rinsing in TBST, a biotinylated goat anti-mouse secondary antibody (1:200 dilution, Vector Laboratories, Burlingame, Calif.) was applied to the tissue sections for 30 minutes at room temperature, followed by the avidin-biotin-peroxidase complex (Vectastain ABC kit; Vector Laboratories, Burlingame, Calif.), according to the manufacturer's protocol. After rinsing in TBST, PrP^{sc} immunoreactivity was visualized using 3,3′-diaminobenzidine (Dakocytomation, Carpinteria, Calif.) as a chromogen, blocked with distilled water. The sections were then counterstained with Meyer's hematoxylin. The PrP^{sc} deposition was evaluated by light microscopy.

### Statistical Analysis.

The survival analysis was performed using the Log-Rank test. To evaluate the differences in the PrP^{sc} among the groups, the results of quantification performed by Western blot analysis were analysed by ANOVA, after checking the assumption of normality and homogeneity of variances.

### Results and Discussion.

All the uninfected untreated or treated animals did not show any clinical signs of disease.

Early clinical signs of prion disease induced by RML strain of scrapie began in untreated mice at ˜130 and ˜180 days post-inoculation respectively ic or ip. Mice initially displayed ruffled coats, assumed kyphotic posture and a tendency to display a straight tail. These early signs of prion disease were followed by ragged or wobbly gait, ataxia and proprioceptive deficits, as evidence by clasped feet when raised by tail. Then they became extremely listless, lethargic and cachectic and they appeared to adopt a frozen posture.

All mice in each infected group, except for two animals in infected ip and treated one, reached terminal disease at a very similar time. At 145^{th} day post-inoculation, the sacrifice of mice which were at the standard clinical end point in groups infected ic either treated or untreated began, and sacrificing continued until the 179^{th} or 180^{th} days post-inoculation in untreated or treated ones. Mice infected ip and not treated were sacrificed between 193^{rd} and 222^{nd} days post-inoculation, while mice infected ip and treated were euthanized between 208^{th} and 250^{th} days. The two mice that weren't sick continued to eat medicated feed until 317^{th} day of trial, then the treatment was discontinued and they were sacrificed forty-four days later, still without clinical signs of disease.

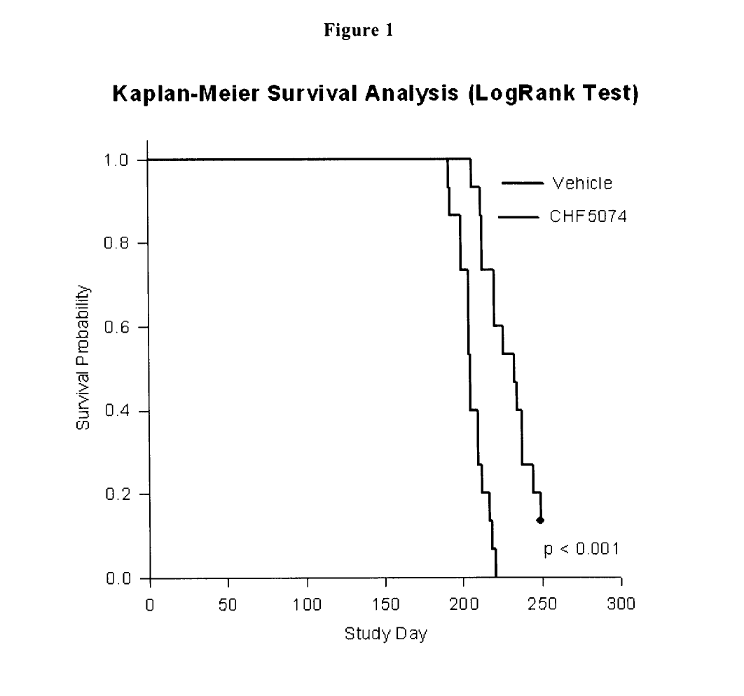

The statistical analysis of survival showed that there was no difference between the survival times of treated or untreated mice infected by intracerebral route (ic). In contrast, (see FIG. 1) there was a significant difference between the survival times of ip infected untreated animals and ip infected treated ones (Log-Rank test: Pvalue=0,000).

Histological, immunohistochemical, and Western blot analysis performed on the brains of mice sacrificed without clinical signs of disease revealed neither the presence of PrP^{sc} nor the neurological lesions associated to prion diseases.

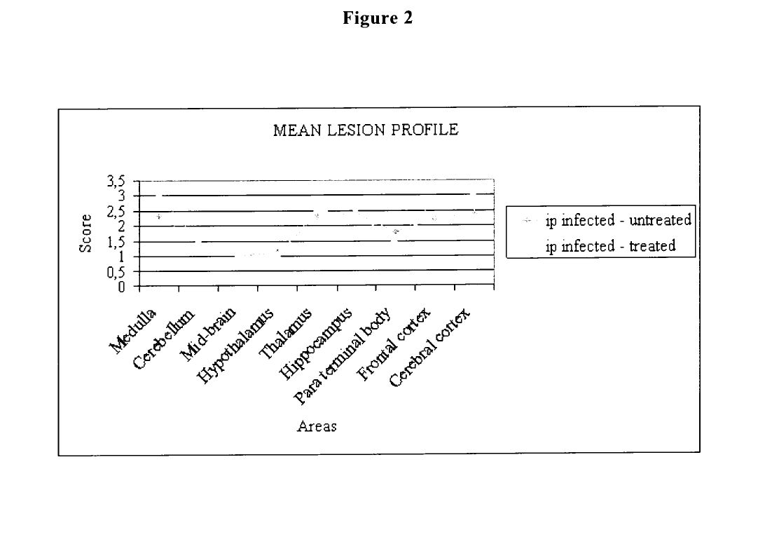

All of the mice sacrificed at the clinical end stage of disease resulted positive to Western blot analysis with the three bands of the pK-resistant PrP corresponding to the di-, mono- and aglycosylated forms of PrP (molecular weight between 30 and 20 kDa), without significant differences in the content of PrP^{sc} among the different groups. Immunohistochemical analysis confirmed the presence of PrP^{sc} in all the samples. The hematoxylin-eosin stain allowed the detection of spongiosis in the nervous tissue and creating a lesion profile of the different encephalic areas, which appears similar in all infected groups, especially in animals infected by intraperitoneal route (see FIG. 2).

Chronic administration of CHF 5074 seems to be safe and well tolerate in CD1 mice, since it did not generate either side effects or toxicity.

The reason that two ip infected mice did not develop the prion disease was very likely because the inoculation failed. They were not sick even after cessation of treatment and showed no neurological damage in laboratory analysis.

The onset and the clinical signs of disease showed by all the other infected animals are compatible with prion disease induced by RML scrapie strain in mice.

Histological analysis, immunohistochemistry and Western blot confirm the presence of a disease induced by prions. The presence of PrP^{sc}, revealed by immunohistochemistry and in the Western blot quantification and the absence of significant differences between the mean lesion profile of the different brain areas confirm that all animals were sacrificed at the same end point.

The significant difference in survival time found between untreated and treated animals in the ip infected groups demonstrates that the chronic administration of CHF 5074 significantly prolongs survival times of CD1 mice infected by intraperitoneal route with the RML scrapie agent, while it has no effect on mice intracerebrally infected.

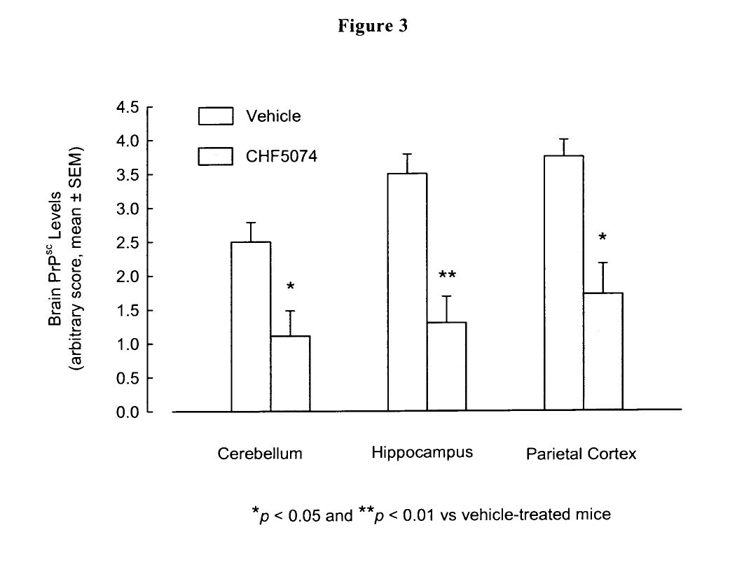

Moreover, immunohistochemical analysis of intraperitoneally infected mice showed significant lower PrP^{sc} deposits in the different brain areas (cerebellum, hippocampus and parietal cortex) of CHF5074-treated animals compared to controls, as shown in FIG. 3.

Without being limited by the theory, on the basis of said findings, it can be reasonably hypothesized that CHF 5074 and close analogs thereof can also be utilized for slowing the progression of prion diseases in humans caused by infection and/or the sporadic forms.

Where a numerical limit or range is stated herein, the endpoints are included. Also, all values and subranges within a numerical limit or range are specifically included as if explicitly written out.

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. A method for preventing and/or treating a prion disease, comprising administering, to a subject in need thereof, an effective amount of a compound of formula (I) 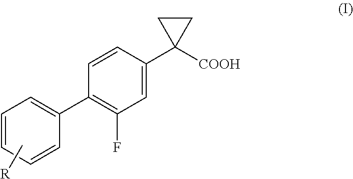
wherein R represents one or more groups, which can be the same or different from each other, independently selected from halogen atoms,
a pharmaceutically acceptable salt, or a prodrug thereof.

2. A method according to claim 1, wherein the halogen atom is chlorine.

3. A method according to claim 1, which comprises administering 1-(3′,4′-dichloro-2-fluorobiphenyl-4-yl)cyclopropanecarboxylic acid.

4. A method according to claim 1, wherein said prion disease is a human prion disease and said subject is a human.

5. A method according to claim 2, wherein said prion disease is a human prion disease and said subject is a human.

6. A method according to claim 3, wherein said prion disease is a human prion disease and said subject is a human.

7. A method according to claim 4, wherein said prion disease is selected from the group consisting of Creutzfeldt-Jacob Disease (CJD), Gerstmann-Straussler-Scheinker (GSS) syndrome, Fatal Familial Insomnia (FFI) and Kuru, and Alpers Syndrome.

8. A method according to claim 5, wherein said prion disease is selected from the group consisting of Creutzfeldt-Jacob Disease (CJD), Gerstmann-Straussler-Scheinker (GSS) syndrome, Fatal Familial Insomnia (FFI) and Kuru, and Alpers Syndrome.

9. A method according to claim 6, wherein said prion disease is selected from the group consisting of Creutzfeldt-Jacob Disease (CJD), Gerstmann-Straussler-Scheinker (GSS) syndrome, Fatal Familial Insomnia (FFI) and Kuru, and Alpers Syndrome.

10. A method according to claim 1, wherein said prion disease is an animal prion disease and said subject is an animal.

11. A method according to claim 2, wherein said prion disease is an animal prion disease and said subject is an animal.

12. A method according to claim 3, wherein said prion disease is an animal prion disease and said subject is an animal.

13. A method according to claim 10, wherein said prion disease is selected from the group of scrapie, transmissible mink encephalopathy (TME), chronic wasting disease (CWD), and bovine spongiform encephalopathy (BSE).

14. A method according to claim 11, wherein said prion disease is selected from the group of scrapie, transmissible mink encephalopathy (TME), chronic wasting disease (CWD), and bovine spongiform encephalopathy (BSE).

15. A method according to claim 12, wherein said prion disease is selected from the group of scrapie, transmissible mink encephalopathy (TME), chronic wasting disease (CWD), and bovine spongiform encephalopathy (BSE).

16. A method according to claim 1, wherein said prion disease is caused by infection.

17. A method according to claim 3, wherein said prion disease is caused by infection.

18. A method according to claim 1, wherein said prion disease is a sporadic form.

19. A method according to claim 3, wherein said prion disease is a sporadic form.
